(19) United States
(12) Patent Application Publication
(10) Pub. No.: US 2007/0208427 A1
(43) Pub. Date: Sep. 6, 2007

(54) PROSTHESIS FOR JOINT REPLACEMENT

(76) Inventors: Marc Gary Davidson, Dublin, OH (US); Bruce Hofer, Lewis Center, OH (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/682,391

(22) Filed: Mar. 6, 2007

Related U.S. Application Data
(60) Provisional application No. 60/779,542, filed on Mar. 6, 2006.

(51) Int. Cl. A61F 2/32; A61F 2/28; A61F 2/30

(52) U.S. Cl. 623/22.15; 623/23.51

(57) ABSTRACT

The invention includes a prosthesis with improved abrasive wear, comprising a composite material. The composite material may comprise an abrasive or superabrasive material dispersed in a continuous matrix of another material. The prosthesis may be formed partially or entirely of composite material, or may be coated with composite material on one or more surfaces. Embodiments include prosthetic joints and articulation surfaces comprising a composite material. Additional embodiments include methods of making a prosthesis comprising a composite material.

### CROSS-REFERENCE TO RELATED APPLICATIONS

This patent application claims priority to U.S. provisional patent application No. 60/779,542 filed Mar. 6, 2006, entitled “Prosthesis for Joint Replacement”, the disclosure of which is incorporated herein by reference in its entirety.

### STATEMENT REGARDING FEDERALLY SPONSORED RESEARCH

Not applicable.

### NAMES OF PARTIES TO A JOINT RESEARCH AGREEMENT

Not applicable.

### SEQUENCE LISTING

Not applicable.

### BACKGROUND

1. Technical Field

The disclosed embodiments generally relate to the field of prosthetics and joint replacement, and more specifically to materials for use in the field and methods of making such materials.

2. Description of the Related Art

Joint replacement surgery is becoming an increasingly common procedure. Surgeries include hip, knee and shoulder replacements. Today 150,000 to 250,000 hip replacements are performed in the U.S. alone each year. Knee and shoulder replacements have also grown in volume. The challenge faced by designers of joint replacement prostheses is to design artificial joints made of biocompatible materials, which mimic those of natural joints. The motion of these joints generally involves the rubbing of two surfaces against one another, such as the rotation of the top or head of the femur in the socket of the pelvis (acetabulum) in a hip joint, such that the surfaces are subject to wear. The wear can, over time, lead to loosening of the lit between these bearing-type surfaces and the introduction of debris into the body. There is evidence that the debris can trigger biological responses that attack the bone and loosen the anchoring of the prostheses in the bone (DeGaspari, J., “Hip Action,” Mechanical Engineering Magazine, December 2004). As people live longer and as joint replacement surgery is used to restore quality of life to lounger, more active patients, the need for a long lasting, low-wear joint replacement prosthesis is increasing.

Total hip replacement was first successfully performed in the early 1960's. Since that time, research has been conducted on the design and materials used in hip replacement prostheses. Designs approved for use in human body by regulatory organizations such as the U.S. FDA include metal or ceramic femoral heads in combination with plastic, metal or ceramic sockets. One of the most common designs utilizes a metal femoral head and a polyethylene socket. Metals most commonly used are cobalt/chrome alloys, titanium and titanium alloys and stainless steels. In some cases the polyethylene socket is backed by metal for support. High molecular-weight and ultra-high molecular weight polyethylene are used to limit wear. However, the polyethylene is relatively soft and wears rather rapidly. Linear wear rates for metal femoral head-on-plastic socket systems are typically 0.1-0.2 millimeters per year (mm/yr). The debris from wear of the socket leads to a loosening of the fit and a condition called osteolysis, where the debris triggers a biological reaction which causes dissolution or breakdown of bone mass. When the ball or head portion of the femoral implant is replaced with a ceramic, such as aluminum oxide or zirconium oxide, wear rates of the ceramic-on-plastic system are lower but still on the order of 0.005 mm/yr and subject to the same problems. Crosslinked varieties of polyethylene have been used to reduce the wear rate, but it is still significant and in the range of 0.005-0.01 mm/yr. Some research has also been done to incorporate ceramics of biological interest, such as hydroxyapatite (HAP), into polymer formulations but to the author's knowledge these materials are not used currently in human joint replacement (see Kannan, S. et al., Trends in Biomaterials and Artificial Organs. 14(2), 2001, p. 30).

Metal femoral heads used in combination with metal sockets (metal-on-metal) offer some advantages but also have disadvantages. The metal-on-metal systems can have 90% lower wear rates. This reduces the loosening of the joint and the introduction of wear particles. However, the metal debris is more biologically active than polyethylene. Increased levels of cobalt, chromium and titanium have been found in the blood and urine of patients with metal-on-metal hip replacements. While not demonstrated, increased levels of these metals in the body could theoretically increase risk of developing cancer. Concerns about ion toxicity also impact doctors' decisions of whether to recommend these types of implant.

Ceramic femoral heads in combination with ceramic sockets (ceramic-on-ceramic) offer some advantages relating to wear and bioreactivity. However, they have significant disadvantages, the most significant of which may be their brittleness. It is estimated that approximately 2% of ceramic-on-ceramic hips break. The ceramic femoral head can fracture at or near the point of attachment to the metal shaft, and such fractures can occur both during attachment and while in service. These implants can also break if inadvertently dropped. Breakage can be very difficult to deal with because it requires surgery to repair or replace, repairs are difficult to make, and it is difficult to remove the debris. Recent improvements, including lowering porosity and decreasing grain size have reduced the risk of fracture but it may still be significant. Another disadvantage of ceramic-on-ceramic implants is long-term mechanical loosening of the implant. Because the ceramics used in these systems have high stiffness or modulus of elasticity compared to the hone they are replacing; the stiffness mismatch between the stiff implant and the flexible bone can cause problems with the implant loosening. In addition, ceramic-on-ceramic joint replacement prostheses are generally expensive.

Some work has been done to apply layers or coatings of conventional ceramic materials, such as alumina, or ceramics of biological interest such as hydroxyapatite (HAP) and tricalcium phosphate (TCP) on metal joint prosthetic surfaces. One such study is described by Armini. et al. in Alternative Rearing Surfaces in Total Joint Replacement ASTM STP 1346, 1998. Others are summarized in Kannan, S. et al., Trends in Biomaterials and Artificial Organs, 14(2), 2001, p3. The goal of these efforts is to improve the abrasion resistance of metal components by adhering a uniform, homogeneous layer of ceramic material to the articulation surface.

The use of diamond and related materials has been proposed as a means of reducing wear in joint replacement prostheses on the basis of diamond's extremely high abrasion resistance. One such approach is the use of diamond-like coatings on the articulation surfaces of a joint replacement. Diamond-like carbon (DLC) is an amorphous form of carbon with 50 to 80% sp3 type carbon to carbon bonding and 1-20% hydrogen. Crystalline diamond is essentially 100% sp3 bonds. DLC's reduced sp3 bond content provides DLC with properties distinctly difierent from crystalline diamond. For example. the hardness of DLC is significantly lower than that of diamond. DLC, depending on processing, has Knoop hardness between 1050 and 2800. This is much less than the 7000 Knoop hardness recorded for diamond. In U.S. Pat. No. 6,626,949, Townley claims a joint implant of specified design with a diamond or diamond-like coating on one or more of the articulation surfaces. U.S. Pat. No. 6,171,343 involves another implant design involving an amorphous carbon coating. Studies have been done on implants coated with DLC to assess biocompatibility (Dowling et al., Diamond Related Mat., 6, 1997, p390) and other performance criteria (Franks et al., in Coombs et al., eds., Nanotechnology in Medicine and Biosciences, Gordon and Breach, 1996, Chapter 9; Santavirta et al., J. Long Term Eff. Med Implants, 9, 1999, p. 67.; Zolynski et al., J. Chem. Vapor Dep., 4, 1996, p. 232.; Lappalainen et al., Clin. Orthop., 352, 1998, p. 118.; Lilley et al., in Doherty ed., Advances in Biomaterials 10; Elsevier, 1992, p. 153; Lappalainen et al., J. Biomed. Mat. Res. B, 66, 2003, p. 410; Lappalainen and Santavirta, Clin. Orthop., 403, 2005, p. 72). While these coatings offer improvements in reducing wear, they face challenges with conforming to complex geometrical surfaces in implants, uniform thickness, quality and adhesion.

A second approach to using diamond in prosthetic joints employs CVD Diamond (CVDD). U.S. Pat. No. 6,517,583 describes the use of CVDD as a counter bearing articulation surface. “Development of CVD diamond coated femoral sliding surfaces for knee joint replacement” (http://univis.uni-erlangen.de/formbot/dsc3Danew_{—}2Fresrep_view_{—}26rprojs_{—}3Dtech_{—}2FIW_{—}2FLWWTM_{—}2Fdev elo_{—}5F85_{—}26dir_{—}3Dtech_{—}2FIW_{—}2FLWWTM_{—}26ref_{—}3Dresrep) describes testing CVD diamond coated titanium knee implants. CVD, unlike DLC, is substantially 100 volume percent crystalline diamond containing over 99% sp3 type bonding. CVDD physical properties are nominally identical to either natural or synthetic diamond. However, the application of CVDD to prosthetic joints is difficult: uniform coatings are difficult to produce, adhesion to metal or ceramics can be unreliable, elastic property mismatches produce high joint stresses, and the coating can be brittle.

A third approach to utilizing diamond in joint replacements employs sintered polycrystalline diamond (PCD). PCD consists of diamond crystals (or grains), typically about ½ micron to about 100 microns in size, fused or sintered together into a continuous diamond matrix at high pressures and high temperatures (HPHT) such as approximately 50 kilobars or more and over 1200° C. PCD is generally about 80% to 95% diamond by volume and is characterized by diamond grains bonded to each other to form a continuous diamond matrix. Interstices between the sintered diamond grains may be filled with a catalyst material used to sinter the grains, another material, or present as voids. Diamicron, Inc. has patented inventions on prosthetic joints, components and fabrication methods involving sintered PCD. (See U.S. Pat. Nos. 6,800,095; 6,793,681; 6,709,463; 6,676,704; 6,610,095; 6,596,225; 6,514,289; 6,497,727; 6,517,583; 6,494,918; 6,488,715; 6,425,992; 6,410,877; 6,402,787; 6,398,815; 6,290,726; and International Patent Application Pub. Nos. WO054612, WO0154627, WO0154613, the disclosures of each of which are incorporated herein by reference).

Sintered PCD has been used for decades for cutting tools, drawing dies, and wear components. It is very costly to produce relative to the ceramic, metal and plastic systems described above. In addition, the size of components which can be fabricated is limited by the working volume of a high-pressure hydraulic press and it most often requires that tile sintered diamond be fused to a substrate material, such as cemented tungsten carbide. These two factors lead to complications in design because the substrate on the PCD articulation surfaces must be attached to the larger prosthesis. PCD is also brittle and subject to chipping or cracking. Due to the HPHT conditions under which it is sintered, there can be significant residual stresses present in the PCD, which can increase the likelihood of chipping and cracking.

There is clearly a need for improved joint replacement prostheses, which have articulation surfaces with lower wear rates (and therefore reduced risk of loosening and osteolysis), and do not have the disadvantages of other low-wear designs, namely high cost, complexity and risk of fracture.

The disclosure contained herein describes attempts to address one or more of the problems described above.

### SUMMARY

The invention generally relates to prosthetics, and more specifically to prosthetics for joint replacement, and to methods of making such prosthetics. One embodiment of the invention is a prosthesis comprising at least two articulation surfaces, such as a head and a socket, wherein at least one of the articulation surfaces comprises an abrasive composite. According to an embodiment, the composite comprises superabrasive or other abrasive material, e.g., superabrasive particles, dispersed in a matrix. In an embodiment, the composite comprises a dispersed abrasive phase and a continuous matrix phase. In another embodiment, the composite comprises a dispersed abrasive particulate and a continuous matrix phase. According to the invention, the abrasive particles do not have significant particle-to-particle bonding.

The abrasive material may comprise superabrasive particles. The matrix may comprise a variety of materials, for example, metal, ceramic, or resin. In an embodiment, the abrasive material adheres to the matrix. In an embodiment, the abrasive material comprises at least about 20% of the composite. According to some embodiments, the matrix is less abrasion resistant than the abrasive phase. The composite used in a prosthesis of the invention may be comprised of physiologically inert material. In an embodiment, the composite contains less than 5% particle-to-particle bonding. In an embodiment, the composite contains, less than 10% particle-to-particle bonding. In an embodiment, the particles within the matrix are diamond particles, and the composite contains no sp3 diamond-to-diamond bonding.

In one embodiment, the composite used in a prosthesis of the invention has a linear wear rate (ASTM G65 or similar standards) of less than about 20 mm³/min. In another embodiment, the linear swear rate for a composite used in a prosthesis of the invention is below about 15 mm³/min. In an embodiment, the linear wear rate for a composite used in a prosthesis of the invention is below about 7 mm³/min. In an embodiment, the composite used in a prosthesis of the invention has a wear rate (Taber) of less than 30 μm/day. In an embodiment, the composite used in a prosthesis of the invention has a wear rate of less than 10 μm/day. In an embodiment, the composite used in a prosthesis of the invention has a coefficient of friction less than 0.5. In an embodiment, the composite used in a prosthesis of the invention has a coefficient of friction less than 0.25. In an embodiment, the composite used in a prosthesis of the invention has a coefficient of friction less than 0.2.

Another embodiment of the invention is a prosthetic joint comprising a first member with an articulating bearing surface, and a second member with a second articulating surface, the second surface conforming to the first articulation surface. One or both members comprise a composite comprising a dispersed abrasive material dispersed in a continuous matrix. One or both articulating surfaces of the joint members may comprise a composite with dispersed abrasive particles in a continuous matrix. The dispersed particles may include superabrasive particles. An embodiment is a prosthetic joint comprising an acetabular cup and a femoral head wherein the cup and head each include an articulating bearing surface, and at least one of the surfaces includes a composite of dispersed abrasive particles in a continuous matrix.

Another embodiment of the invention is an implantable prosthesis, comprising an articulated joint with bearing surfaces wherein at least one of the bearing surfaces comprises a composite containing a distributed hard phase within a continuous matrix. The composite may contain abrasive, superabrasive, or a combination of the former particles dispersed in a matrix material. The matrix may comprise, for example, metal, ceramic, or resin. The abrasive, superabrasive, or combined particles may be adhered to the matrix material. The abrasive, superabrasive, or combined particles may comprise, in total, at least about 20% of the composite.

An embodiment of the invention is an articulation surface for use in a prosthetic joint, the comprising a composite. The surface may comprise a composite with a dispersed abrasive phase and a continuous matrix phase. According to another embodiment, the surface comprises a structural substrate and a composite coating, wherein the composite coating comprises a dispersed abrasive particulate and a continuous matrix phase, and wherein the continuous matrix phase adheres to the abrasive particulate and to the substrate. The surface abrasive may comprise superabrasive particles.

The invention further includes methods of making a prosthesis comprising a composite, as further described herein.

### BRIEF DESCRIPTION OF THE DRAWINGS

FIG. 1 depicts a cross-section of a Ni—P/diamond composite coating.

### DETAILED DESCRIPTION

Before the present methods, systems and materials are described, it is to be understood that this disclosure is not limited to the particular methodologies, systems and material described, as these may vary. It is also to be understood that the terminology used in the description is for the purpose of describing the particular versions or embodiments only, and is not intended to limit the scope, for example, as used herein and in the appended claims, the singular forms “a,” “an,” and “the” include plural references unless the context clearly dictates otherwise. In addition, the word “comprising” as used herein is intended to mean “including but not limited to.” Unless defined otherwise, all technical and scientific terms used herein have the same meanings as commonly understood by one of ordinary skill in the art.

Embodiments described herein exploit the beneficial wear-resistant and low friction properties of “superabrasive” materials in joint replacement prostheses without one or more of the complexities, shortcomings or high costs associated with the approaches previously proposed. Superabrasives are materials having a hardness of at least about 2000 Knoop or higher, such as diamond and cubic boron nitride. Superabrasives are distinguished from “conventional” hard or abrasive particles such as alumina, zirconia, silicon carbide tungsten carbide and ceramics of biological interest by their extreme hardness and abrasion resistance

In one embodiment of the invention, composite materials are provided comprising abrasive or superabrasive particles dispersed or distributed in a matrix of another material. Abrasive and superabrasive particles used in the invention are generally less than about 500 micron in diameter and preferably less than about 100 microns. The composite materials of the invention may be used to coat some or all of the articulation surfaces of a joint replacement prosthesis, or may be used to form the entire prosthesis itself.

The continuous matrix may be any material such as metal, a metal alloy, polymer, conventional ceramic, non-covalent ceramic non-carbide ceramic, glass, composite or combinations thereof. Preferably, the abrasion resistance of a matrix material is lower than that of a superabrasive. Preferably, the material selected is physiologically inert. Superabrasive particles of a composite may be present in any concentration or volume fraction but generally at least about 20% by volume, to provide the desired wear resistance. Exemplary concentration ranges of superabrasive particles include about 20 to about 50 volume percent, about 25 to about 50 volume percent, about 25 to about 40 volume percent, about 50 to about 70 volume percent, and about 15 to about 70 volume percent.

According to an embodiment, a composite used in a prosthesis of the invention has a linear wear rate (ASTM G65 or similar standards) of less than about 20 mm³/min. The linear wear rate for the composite used in a prosthesis of the invention may be below about 15 mm³/min, or may be below about 7 mm/³min. In an embodiment, the composite used in a prosthesis of the invention has a Taber wear rate of less than 30 μm/day. In an embodiment, the composite used in a prosthesis of the invention has a wear rate of less than 10 μm/day. In an embodiment, the composite used in a prosthesis of the invention has a coefficient of friction less than 0.5. In an embodiment, the composite used in a prosthesis of the invention has a coefficient of friction less than 0.25. In an embodiment, the composite used in a prosthesis of the invention has a coefficient of friction less than 0.2.

In an embodiment, an abrasive or superabrasive particle adheres to the matrix. While not wishing to be bound by theory, the particle in this embodiment may be adhered to the matrix through any combination of mechanical bonding, primary chemical bonding, secondary interactions, such as for example, but not limited to, dispersion forces, van der Waals interactions, hydrogen bonding and the like. Particles may be coated to improve their adherence to the matrix material or to prevent the matrix material from chemically reacting with the particles. More than one particle type may be used in a single composite. The matrix may also contain other dispersed or continuous phases to provide other functions, including fillers, reinforcing whiskers or fibers, bioactive materials to improve biocompatibility, non-superabrasives or ceramics of biological interest, lubricants or other materials.

The composite material, comprising discrete superabrasive particles and continuous matrix can be implemented as the entire component, a subcomponent in an assembly, or as a layer or coating on a backing material. Thee composite and/or component can be post-processed to further improve performance. Post-processing can include bulk treatments such as heat treatment or annealing or surface treatments such as lapping, polishing, or coating with other materials, such as lubricants or packing materials.

Unlike the prior art, the composite materials described herein do not contain significant amounts of abrasive-to-abrasive (such as diamond-to-diamond) bonding; rather, the particles are substantially dispersed, i.e. discrete, within a continuous matrix. As such, the abrasive (including superabrasive) particles of the composite comprise a discontinuous phase within a continuous matrix and less than 25% of the particles form particle-to-particle bonds. In an embodiment, the composite contains less than 10% particle-to-particle bonding. In an embodiment, the composite contains less than 5% particle-to-particle bonding. In an embodiment, the particles within the matrix are diamond particles, and the composite contains no sp3 diamond-to-diamond bonding.

The use of such a composite material provides improved wear resistance and associated benefits while reducing the complexity, cost and undesirable properties (such as brittleness) of prior art solutions. Each of the prior art solutions, with the exception of ceramic-loaded polymers, involve single materials or sintered composites where there is grain-to-grain bonding of the abrasive material, making the abrasive material largely a continuous phase for example, the prior art involving sintered PCD has substantial bonding between the diamond grains, forming a continuous diamond matrix. Similarly ceramic articulation surfaces involve sintering of ceramic grains to form the solid component. In both of these cases, the sintering process adds cost and results in undesirable properties. Other solutions such as DLC coatings, CVD diamond coatings, ceramic coatings, metal and polymeric components involve single materials or alloys on the articulation surfaces. Ceramic-loaded polymers have distributed ceramic particles, but work in this area has been restricted to ceramics with low abrasion resistance and in most cases ceramics of biological interest.

One embodiment of the invention involves coating one or more surfaces of metal joint replacement components, e.g., the ball and/or socket (i.e., femoral head and acetabular cup) of a hip replacement prosthesis, with a composite coating comprised of a metal matrix and superabrasive particles such as diamond. Alternatively, the prosthesis may be for the shoulder, knee or other joint. Such a coating can be applied by any number of methods including electroless or electrolytic plating, thermal spray methods, vapor deposition methods, anodizing, etc. With appropriate selection of application technique and surface preparation, the metallic matrix of the coating will adhere strongly to the metallic component body, thereby overcoming the limitation of some DLC and CVD diamond coatings. In some embodiments, the composite contains about 30 to about 40 volume percent diamond and/or other superabrasive. In other embodiments, it contains up to 70 volume percent diamond and/or other superabrasive. Other superabrasive concentrations are possible, as described herein.

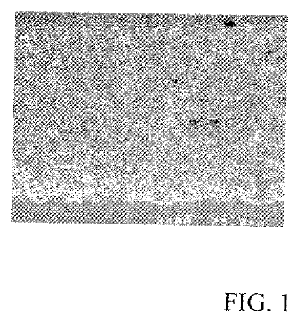

In some cases, the metal matrix can be chosen to be of the same or similar composition as the base component. For example, a titanium-diamond composite coating could be applied to a titanium base of the type currently used in joint prostheses or a composite of diamond, cobalt and/or chromium could be applied to a cobalt/chrome component. One example of a metal matrix-abrasive composite coating is a Ni-diamond composite prepared using electroless plating methods described in the patent literature (U.S. Pat. Nos. 3,936,577; 4,997,686; 5,145,517; 5,300,330; 5,863,616; 6,306,466, the disclosures of each of which are incorporated herein by reference) and related literature (e.g., Electroless Nickel Coatings-Diamond Containing, R. Barras et al., Electroless Nickel Conference, November 1979, Cincinnati, Ohio). The micrograph in FIG. 1 shows a composite coating with Nickel-phosphorus as the metal matrix and diamond as the distributed phase. Other metal matrices may include, without limitation, electroless copper, cobalt, or silver. Examples of electrolytic processes may include chromium, nickel, platinum, or iron.

Another approach to forming a metal-superabrasive composite for the articulation surfaces of a joint replacement prosthesis is to embed a superabrasive in a metal component or portion of a metal component. Embedding may occur while the component is being formed or as a post-formation treatment. For example, a superabrasive may be incorporated in a metal matrix as it is cast, or incorporated as a component in powder metal processes. For certain superabrasive materials or coated superabrasives that can withstand the temperatures and chemical environment, it is possible to introduce the superabrasive particles to the molten metal during casting. A more practical and probably more broadly applicable approach is to use powdered metals. The powdered metals can be blended with superabrasive particles and molded into the articulation surface, for example by injection or compression molding. Elevated temperatures will be required to sinter the metal grains and form the continuous or semi-continuous metal matrix around the superabrasive particles. The temperature can be applied either while pressure is being applied as in hot isostatic pressing or alter forming a “green body” in a so-called free sintering operation. Options include forming the entire component as a metal-superabrasive composite, applying a metal-superabrasive composite layer on the articulation surface of a component base, or even applying a graded matrix-superabrasive composite in which the concentration of the abrasive may vary with position in the component. In some embodiments, the coating thickness could be between about 20 and about 500 microns thick. Other thicknesses are possible.

Another embodiment includes a composite material with a polymeric matrix and distributed superabrasive particles. Diamond or cubic boron nitride particles can be introduced to the polymer matrix in numerous ways including but not limited to blending of resin and superabrasive particles prior to compression molding, compounding the superabrasive into molten resin for injection molding, solution casting, or blending prior to curing or crosslinking. The resin can be of any type including filled, unfilled or reinforced thermoplastics, thermosets, cross-linked polymers, epoxies, etc. The composite can comprise the entire component with the superabrasive distributed throughout it, a layer adhered to a backing of other material a thin integral layer or coating on a substrate, or a component with the superabrasive concentration increasing towards the articulation surface. Graded or layered structures can be formed, for example, by layering powders, by co-injection of superabrasive-containing and non-superabrasive containing melts, or by introducing the polymer or solution into a mold after distributing the superabrasive particulate. Machining, grinding, shaping, or otherwise post-processing man be needed to convert the composite/component into its final form for use in the prostheses.

Another embodiment includes a composite material with a ceramic matrix and distributed superabrasive particles. This composite can be fabricated using any process for fabricating a ceramic body by blending in the superabrasive particles with the ceramic powder prior to forming a green body and/or sintering. Again, the superabrasive composite can be the entire joint replacement component, a concentrated layer on the articulation surface of the component, or a graded composite with higher concentrations of superabrasive particles. While this composite may have similar limitations to existing ceramics with regard to brittleness and fracture, the superabrasive has the potential to improve wear resistance further.

Another embodiment is a composite material comprised of superabrasive particles distributed in a mental-ceramic co-composite, such as those developed and marketed by Excera Materials Inc. under the ONNEX name. These matrix materials are co-continuous composites of ceramic and metal with domain widths on the order of 10 micrometers (μm). The superabrasive can be introduced with the metal and ceramic powders prior to forming the green body. Again the superabrasive-containing composite can be the bulk component a layer on the articulation surface or a graded composite with superabrasive concentration increasing toward the articulation surface.

The approaches described herein can be extended to conventional abrasives. e.g., to improve abrasion resistance of metal articulation surfaces of joint replacement prostheses with distributed abrasive grains. For example, a nickel matrix with silicon carbide abrasive particles dispersed therein may be used in the applications described herein. The invention includes, therefore, prostheses with improved wear, formed or coated by composites comprising abrasive materials dispersed in a continuous matrix. Exemplary composite materials may have about 20 volume percent abrasive or more, with about 25 to about 40 volume percent as a preferred range.

The invention is applicable to human prosthetics but may also be utilized in veterinary prosthetics. The invention further includes methods of making the disclosed prosthetic devices.

### EXAMPLES

In one set of tests, abrasive wear was measured by abrading test specimens with a grit of controlled size and composition. The test was based upon Procedure C of the ASTM G65 standard, in which a test specimen is pressed against a rotating wheel (with rubber rim) while a controlled flow of grit introduced to the gap between the wheel and the test specimen in the direction of wheel rotation. A DUOCOM dry abrasion tester was used at 200 rotations per minute (rpm) wheel speed and a load of 130 Newtons. AFS 50-70 silica sand of about 200 to about 300 μm grain size was fed to the gap at 30 grams/min for 30 seconds.

Samples tested were 304 stainless steel coupons electrolessly coated with Ni—P or Ni—P/diamond composites of varying grain sizes, diamond volume fractions and phosphorous content. The coating method used to coat the diamond-free Ni coating was standard electroless Ni plating; coating of the Ni—P/diamond composites is described in Electroless Nickel Coatings-Diamond Containing, R. Barras et al., Electroless Nickel Conference. November 1979, (Cincinnati, Ohio and U.S. Pat. Nos. 3,936,577; 4,997,686l 5,145,517; 5,300,330; 5,863,616; and 6,306,466. All Ni—P and Ni—P/composite test coupons were heat treated at about 400C.° for one hour in nitrogen to improve their abrasion resistance. Tested for comparison were the bare stainless steel coupon, and coupons coated with hard chrome, Stellite and an alumina-titania (13 weight-%>titania) coating. Hard chrome was deposited by standard plating methods to a thickness of 200 μm Stellite-6 is a Co-based hardfacing deposited by welding to a thickness of 500 μm and alumina-titania is deposited by plasma spraying (with a bond coat thickness of 10 μm of Ni—Al—B—Si) to a thickness of about 100 μm. All samples were cleaned in an ultrasonic bath of acetone for 10 minutes, dried and weighed before the test.

Following the dry abrasion testing, the samples were again cleaned in the ultrasonic bath for 10 minutes dried and reweighed. Abrasive wear was determined based on loss of mass, converted to volume using density of the coating, and reported in cubic millimeters per minute (mm³/min.) In all cases, the wear test was completed before the coating depth was penetrated.

The Ni—P coating (4% P) wore at a rate of 28.3 mm³/min, while all of the Ni—P/diamond composites (4 and 9% P) wore at a rate of 2.4 to 6.5 mm³/min. A coating with 30 volume-percent diamond with mean grain size of 2 um was the best performing on average with 2.9 mm³/min average wear. Coatings with 25 volume-percent diamond of 0.25 μm grain size also performed well with an average wear rate of 4.1 mm³/min. Coating with 40 volume-% diamond of 8 μm mean size averaged 6.2 mm³/min. Higher phosphorus levels improved abrasion resistance of the composite coatings somewhat, while there was no effect of coating thickness (between 50 and 200 μm). Nonetheless, the data clearly show a 4 to 10× improvement in abrasion resistance between a mental surface (Ni) and a Ni/diamond composite of the same matrix material. For comparison, bare stainless steel whore at more than 60 mm³/min and the Stellite coating more than 50 mm³/min. The ceramic alumina-titania coating and hard chrome coatings showed improved wear resistance relative to the Ni—P at approximately 22 and 7 mm³/min, respectively, however neither performed nearly as well as any of the Ni—P/diamond composites. In fact, the best Ni—P/composite was roughly 7× better than the ceramic coating.

A pin-and-disc tribometer was used to measure sliding wear and friction. The instrument, from CSM Instruments SA, has a sample holder where a 55 mm diameter disc (coated or uncoated), with a height of 5-10 mm, can be mounted and screwed to the instrument. The other contact material was a pin of 6 mm diameter and 10 mm height. The disc can be rotated at a speed of 0-500 rpm, while the pin is stationary. The pin holder holds the pin tightly at the button against the disc. The pin was loaded with a load of 10N for all tests. Tests were run at 0.5 m/sec sliding velocity for 2000 meters. A trace of friction coefficient against time and sliding distance was obtained through the computer interface. The wear loss of disc and pin was obtained by measuring the weight before and after the test. The samples were ultrasonically cleaned in acetone before the weight measurements were done. The coatings described above were used in this test.

When both pin and disc were coated with the identical material dry wear factors on the disc (reported in 10E-5 mm³/Nm) were 3.4 for Ni—P and 1.1 to 1.7 for the Ni—P/diamond composites a 2 to 3× improvement. Wear factors for the pin were also better, 0.44 for the Ni—P and 0.15 to 0.30 for the diamond-containing composite. The Ni—P/diamond composites also outperformed the bare 304 stainless steel, alumina-titania, and Stellite which had disc wear rates of 30, 151, and 14. Hard chrome was competitive at 1.6, but based on the improvement seen by introducing diamond to the Ni—P coating one would expect similar improvement if diamond were present in a composite chrome/diamond composite coating. Pin wear for these alternative materials showed the same trend.

The Ni—P/diamond composites also had much lower coefficients of friction. Ni—P/diamond with 2 and 8 μm grain sizes had coefficients of friction of 0.17 and 0.12, respectively, versus Ni—P at 0.55, hard chrome at 0.72, Stellite at 0.3, alumina-titania at 0.76 and bare 304 stainless steel at 0.63. Clearly the diamond-containing composites reduce the friction between sliding components.

Testing of wear in another standard test, a Taber test, was also performed to compare Ni—P coating to the Ni—P/diamond composites. A model 5130 Taber tester was used with 5000 cycles with a 1000-gram load and CS-10 wheel. Wear rate for the composite with 8 μm grain size was only 0.2 μm/day, with 2 μm grains at 4 μm/day and with 0.25 μm grains at 7 μm/day. By comparison the Ni—P coating wore at 30 μm/day. Once again, these data demonstrate the improved wear resistance provided by the distributed superabrasive grains in the matrix.

It will be appreciated that various of the above-disclosed and other features and functions, or alternatives thereof, may be desirably combined into many other different systems or applications. Various presently unforeseen or unanticipated alternatives, modifications, variations or improvements therein may be subsequently made by those skilled in the art which are intended to be encompassed by the following claims.

What is claimed is:

1. A prosthesis, comprising:
at least two articulation surfaces;
wherein at least one of the articulation surfaces comprises an abrasive composite comprising superabrasive particles dispersed in a matrix material.

2. The prosthesis of claim 1, wherein the matrix material comprises metal, ceramic, or resin.

3. The prosthesis of claim 1, wherein the superabrasive particles are adhered to the matrix material.

4. The prosthesis of claim 1, wherein the superabrasive particles comprise at least about 20% of the composite.

5. An articulation surface for use in a prosthetic joint, the articulation surface comprising:
a composite with a dispersed abrasive phase and a continuous matrix phase, said matrix being less abrasion resistant than the abrasive phase.

6. The surface of claim 5, wherein the matrix is comprised of a physiologically inert material.

7. The surface of claim 5, wherein the abrasive phase comprises superabrasive particles.

8. An articulation surface for a prosthetic joint comprising:
a structural substrate; and
a composite coating;
wherein the composite coating comprises a dispersed abrasive particulate and a continuous matrix phase, and wherein the continuous matrix phase adheres to the abrasive particulate and to the substrate.

9. The articulation surface of claim 8 wherein the abrasive comprises a superabrasive with hardness greater than 2000 Knoop.

10. A prosthetic joint comprising:
a first member with an articulating bearing surface;
a second member with a second articulating surface, the second surface conforming to the first articulation surface;
wherein one or both articulating surfaces comprise a composite with dispersed particles in a continuous matrix.

11. The joint of claim 10 wherein the particles include superabrasive particles.

12. A prosthetic joint comprising:
an acetabular cup; and
a femoral head;
wherein the cup and head each include an articulating bearing surface;
wherein at least one of the surfaces includes a composite of dispersed abrasive particles in a continuous matrix.

13. An implantable prosthesis, comprising:
an articulated joint with bearing surfaces;
wherein at least one of the bearing surfaces comprises a composite containing a distributed hard phase within a continuous matrix.

14. The prosthesis of claim 11, wherein the composite contains superabrasive particles dispersed in a matrix material.

15. The prosthesis of claim 13, wherein the matrix comprises metal, ceramic, or resin.

16. The prosthesis of claim 14, wherein the superabrasive particles are adhered to the matrix material.

17. The prosthesis of claim 14, wherein the superabrasive particles comprise at least about 20% of the composite.

18. A prosthesis, comprising:
a head; and
a socket;
wherein at least one surface of the head, the socket, or both the head and socket is coated with an abrasive composite comprising superabrasive particles and a matrix material, wherein the superabrasive particles do not have significant particle-to-particle bonding.

19. The prosthesis of claim 18, wherein the superabrasive particle is diamond, and the composite contains no sp3 diamond-to-diamond bonding.
